(19) United States
(12) Patent Application Publication
(10) Pub. No.: US 2007/0073136 A1
(43) Pub. Date: Mar. 29, 2007

(54) Bone milling with image guided surgery

(76) Inventor: Robert Metzger, Wakarusa, IN (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/373,899

(22) Filed: Mar. 12, 2006

Related U.S. Application Data
(60) Provisional application No. 60/717,550, filed on Sep. 15, 2005.

(51) Int. Cl. A61B 5/05

(52) U.S. Cl. 600/407

(57) ABSTRACT

A method for resurfacing a bone at a surgical site during a surgical navigation procedure is provided. The method comprises providing a tracking system and a surgical instrument having a tracking array, the tracking array being identified and tracked by the tracking system. The surgical instrument is moved relative to a bone while the tracking system tracks the position of the surgical instrument, and the relative movement is projected on an image of the bone. The projected image is viewed as the surgical instrument is moved relative to the bone to determine when the surgical instrument is positioned at the surgical site. The surgical instrument is used to make a hole in the bone at the surgical site, and a guide pin is inserted into the hole.

### RELATED APPLICATIONS

This application claims priority to U.S. provisional application Ser. No. 60/717,550, filed Sep. 15, 2005, the disclosure of which is expressly incorporated by reference herein.

### FIELD OF THE INVENTION

The present teachings relate to surgical navigation and more particularly to a method of resurfacing a bone with a surgical navigation system.

### BACKGROUND

Surgical navigation systems, also known as computer assisted surgery and image guided surgery, aid surgeons in locating patient anatomical structures, guiding surgical instruments, and implanting medical devices with a high degree of accuracy. Surgical navigation has been compared to a global positioning system that aids vehicle operators to navigate the earth. A surgical navigation system typically includes a computer, a tracking system, and patient anatomical information. The patient anatomical information can be obtained by using an imaging mode such as fluoroscopy, computer tomography (CT) or by simply defining the location of patient anatomy with the surgical navigation system. Surgical navigation systems can be used for a wide variety of surgeries to improve patient outcomes.

To successfully implant a medical device, surgical navigation systems often employ various forms of computing technology, as well as utilize intelligent instruments, digital touch devices, and advanced 3-D visualization software programs. All of these components enable surgeons to perform a wide variety of standard and minimally invasive surgical procedures and techniques. Moreover, these systems allow surgeons to more accurately plan, track and navigate the placement of instruments and implants relative to a patient's body, as well as conduct pre-operative and intra-operative body imaging.

Because of the complexity of many image guided surgery procedures, surgeons often use a variety of instruments during a single procedure. Many of these instruments require invasive application, thereby increasing the patient's risk of infection and/or embolism. For instance, in many surgical bone resection procedures, the surgeon invasively anchors an intramedullary (“IM”) referencing rod/guide directly into the internal portion of a patient's bone. Such invasive actions increase the complexity of the procedure and often slow the patient's recovery. Accordingly, it would be desirable to overcome these and other shortcomings of the prior art.

### SUMMARY OF THE INVENTION

The present teachings provide a method of resurfacing a bone during a surgical navigation procedure that reduces the need to use invasive instruments and improves the accuracy to which the bone is cut.

In one exemplary embodiment, the present teachings provide a method of resurfacing a bone at a surgical site during a surgical navigation procedure. The method comprises providing a tracking system and a surgical instrument having a tracking array, the tracking array being identified and tracked by the tracking system. The surgical instrument is moved relative to a bone while the tracking system tracks the position of the surgical instrument, and the relative movement of the surgical instrument is projected on an image of the bone. The projected image is viewed as the surgical instrument is moved relative to the bone to determine when the surgical instrument is positioned at the surgical site, and the surgical instrument is used to make a hole in the bone at the surgical site. A guide pin is then inserted into the hole.

### BRIEF DESCRIPTION OF THE DRAWINGS

The above-mentioned aspects of the present teachings and the manner of obtaining them will become more apparent and the teachings will be better understood by reference to the following description of the embodiments taken in conjunction with the accompanying drawings, wherein:

FIG. 1 is a perspective view of an exemplary operating room setup in a surgical navigation embodiment in accordance with the present teachings;

FIG. 2 is an exemplary block diagram of a surgical navigation system embodiment in accordance with the present teachings;

FIG. 3 is an exemplary surgical navigation kit embodiment in accordance with the present teachings;

FIG. 4 is a flowchart illustrating the operation of an exemplary surgical navigation system in accordance with the present teachings;

FIG. 5 is a perspective view of an exemplary surgical reamer instrument in accordance with the present teachings shown aligned with a surgical guide pin;

FIG. 6 is a sectional view of the exemplary surgical reamer instrument of FIG. 5 taken along line 5A-5A and shown positioned over the surgical guide pin; and

FIGS. 7A-7G are perspective views illustrating a bone undergoing an exemplary milling process in accordance with the present teachings.

Corresponding reference characters indicate corresponding parts throughout the several views.

### DETAILED DESCRIPTION

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

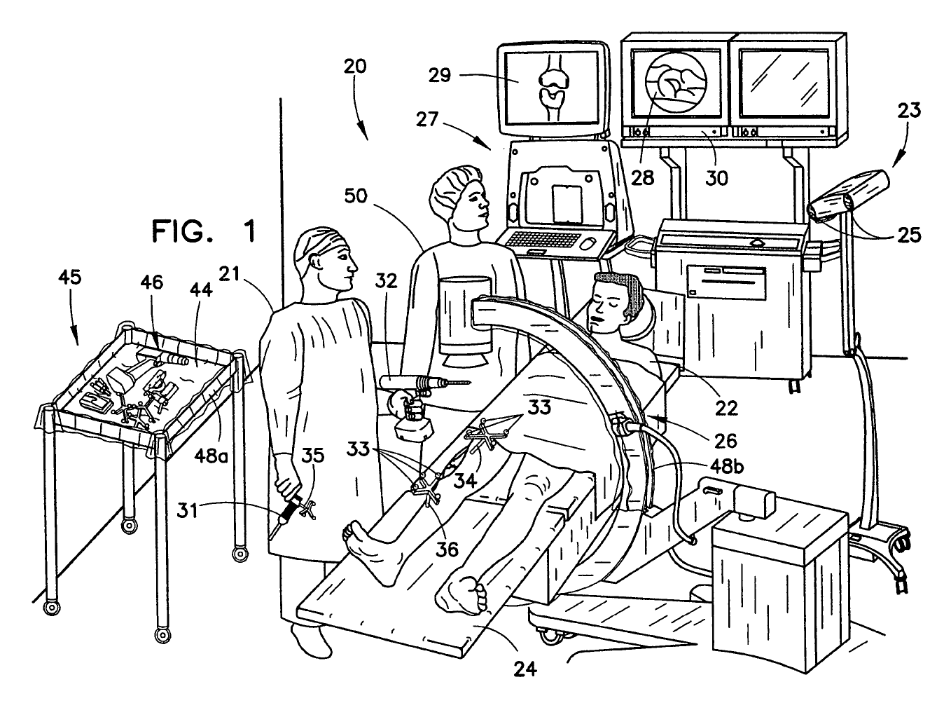

FIG. 1 shows a perspective view of an operating room with surgical navigation system 20. Surgeon 21 is aided by the surgical navigation system in performing knee arthroplasty, also known as knee replacement surgery, on patient 22 shown lying on operating table 24. Surgical navigation system 20 has a tracking system that locates arrays and tracks them in real-time. To accomplish this, the surgical navigation system includes optical locator 23, which has two CCD (charge couple device) cameras 25 that detect the positions of the arrays in space by using triangulation methods. The relative location of the tracked arrays, including the patient's anatomy, can then be shown on a computer display (such as computer display 27 for instance) to assist the surgeon during the surgical procedure. The arrays that are typically used include probe arrays, instrument arrays, reference arrays, and calibrator arrays. The operating room includes an imaging system such as C-arm fluoroscope 26 with fluoroscope display image 28 to show a real-time image of the patient's knee on monitor 30. The tracking system also detects the location of surgical instruments, such as drill guide 31 and/or surgical tools, such as drill 32, as well as reference arrays 34, 36, which are attached to the patient's femur and tibia. By knowing the location of markers 33 attached to the surgical instruments, the tracking system can detect and calculate the position of the instruments in space. The operating room also includes instrument cart 45 having tray 44 for holding a variety of surgical instruments and arrays 46. Instrument cart 45 and C-arm 26 are typically draped in sterile covers 48a, 48b to eliminate contamination risks within the sterile field.

The surgery is performed within a sterile field, adhering to the principles of asepsis by all scrubbed persons in the operating room. Patient 22, surgeon 21 and assisting clinician 50 are prepared for the sterile field through appropriate scrubbing and clothing. The sterile field will typically extend from operating table 24 upward in the operating room. Typically both the computer display and fluoroscope display are located outside of the sterile field.

A representation of the patient's anatomy can be acquired with an imaging system, a virtual image, a morphed image, or a combination of imaging techniques. The imaging system can be any system capable of producing images that represent the patient's anatomy such as a fluoroscope producing x-ray two-dimensional images, computer tomography (CT) producing a three-dimensional image, magnetic resonance imaging (MRI) producing a three-dimensional image, ultrasound imaging producing a two-dimensional image, and the like. A virtual image of the patient's anatomy can be created by defining anatomical points with surgical navigation system 20 or by applying a statistical anatomical model. A morphed image of the patient's anatomy can be created by combining an image of the patient's anatomy with a data set, such as a virtual image of the patient's anatomy. Some imaging systems, such as C-arm fluoroscope 26, can require calibration. The C-arm can be calibrated with a calibration grid that enables determination of fluoroscope projection parameters for different orientations of the C-arm to reduce distortion. A registration phantom can also be used with a C-arm to coordinate images with the surgical navigation application program and improve scaling through the registration of the C-arm with the surgical navigation system. A more detailed description of a C-arm based navigation system is provided in James B. Stiehl et al., Navigation and Robotics in Total Joint and Spine Surgery, Chapter 3: C-Arm-Based Navigation, Springer-Verlag (2004).

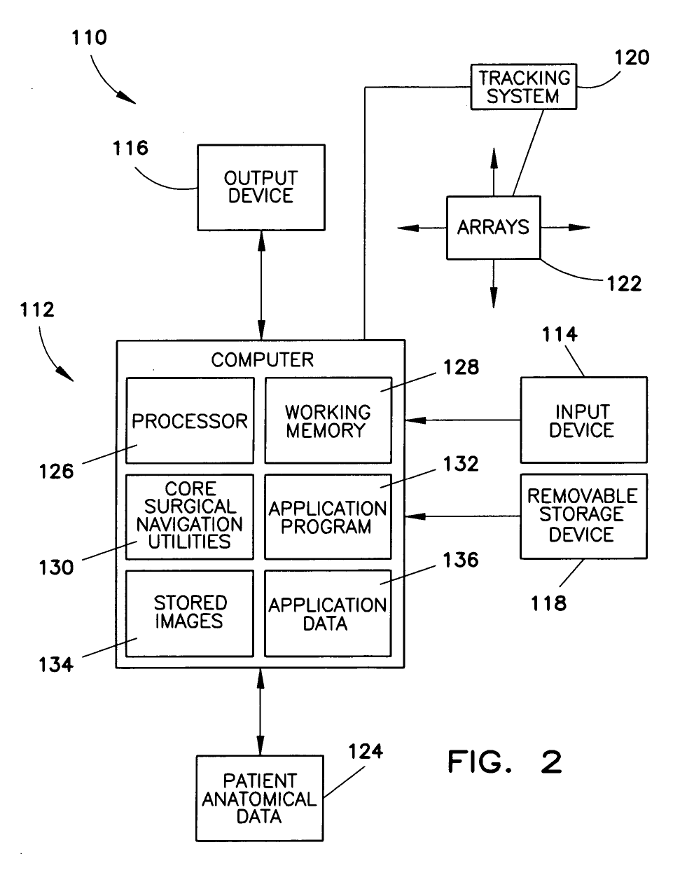

FIG. 2 is a block diagram of an exemplary surgical navigation system embodiment in accordance with the present teachings, such as an Acumen™ Surgical Navigation System, available from EBI, L.P., Parsipanny, N.J. USA, a Biomet Company. The surgical navigation system 110 comprises computer 112, input device 114, output device 116, removable storage device 118, tracking system 120, arrays 122, and patient anatomical data 124, as further described in the brochure Acumen™ Surgical Navigation System, Understanding Surgical Navigation (2003) available from EBI, L.P. The Acumen™ Surgical Navigation System can operate in a variety of imaging modes such as a fluoroscopy mode creating a two-dimensional x-ray image, a computer-tomography (CT) mode creating a three-dimensional image, and an imageless mode creating a virtual image or planes and axes by defining anatomical points of the patient's anatomy. In the imageless mode, a separate imaging device such as a C-arm is not required, thereby simplifying set-up. The Acumen™ Surgical Navigation System can run a variety of orthopedic applications, including applications for knee arthroplasty, hip arthroplasty, spine surgery, and trauma surgery, as further described in the brochure “Acumen™ Surgical Navigation System, Surgical Navigation Applications” (2003), available from EBI, L.P. A more detailed description of an exemplary surgical navigation system is provided in James B. Stiehl et al., Navigation and Robotics in Total Joint and Spine Surgery, Chapter 1: Basics of Computer-Assisted Orthopedic Surgery (CAOS), Springer-Verlag (2004).

Computer 112 can be any computer capable of properly operating surgical navigation devices and software, such as a computer similar to a commercially available personal computer that comprises a processor 126, working memory 128, core surgical navigation utilities 130, an application program 132, stored images 134, and application data 136. Processor 126 is a processor of sufficient power for computer 112 to perform desired functions, such as one or more microprocessors. Working memory 128 is memory sufficient for computer 112 to perform desired functions such as solid-state memory, random-access memory, and the like. Core surgical navigation utilities 130 are the basic operating programs, and include image registration, image acquisition, location algorithms, orientation algorithms, virtual keypad, diagnostics, and the like. Application program 132 can be any program configured for a specific surgical navigation purpose, such as orthopedic application programs for unicondylar knee (“uni-knee”), total knee, hip, spine, trauma, intramedullary (“IM”) nail/rod, and external fixator. Stored images 134 are those recorded during image acquisition using any of the imaging systems previously discussed. Application data 136 is data that is generated or used by application program 132, such as implant geometries, instrument geometries, surgical defaults, patient landmarks, and the like. Application data 136 can be pre-loaded in the software or input by the user during a surgical navigation procedure.

Output device 116 can be any device capable of creating an output useful for surgery, such as a visual output and an auditory output. The visual output device can be any device capable of creating a visual output useful for surgery, such as a two-dimensional image, a three-dimensional image, a holographic image, and the like. The visual output device can be a monitor for producing two and three-dimensional images, a projector for producing two and three-dimensional images, and indicator lights. The auditory output can be any device capable of creating an auditory output used for surgery, such as a speaker that can be used to provide a voice or tone output.

Removable storage device 118 can be any device having a removable storage media that would allow downloading data, such as application data 136 and patient anatomical data 124. The removable storage device can be a read-write compact disc (CD) drive, a read-write digital video disc (DVD) drive, a flash solid-state memory port, a removable hard drive, a floppy disc drive, and the like.

Tracking system 120 can be any system that can determine the three-dimensional location of devices carrying or incorporating markers that serve as tracking indicia. An active tracking system has a collection of infrared light emitting diode (ILEDs) illuminators that surround the position sensor lenses to flood a measurement field of view with infrared light. A passive system incorporates retro-reflective markers that reflect infrared light back to the position sensor, and the system triangulates the real-time position (x, y, and z location) and orientation (rotation around x, y, and z axes) of an array 122 and reports the result to the computer system with an accuracy of about 0.35 mm Root Mean Squared (RMS). An example of a passive tracking system is a Polaris® Passive System and an example of a marker is the NDI Passive Spheres™, both available from Northern Digital Inc. Ontario, Canada. A hybrid tracking system can detect active and active wireless markers in addition to passive markers. Active marker based instruments enable automatic tool identification, program control of visible LEDs, and input via tool buttons. An example of a hybrid tracking system is the Polaris® Hybrid System, available from Northern Digital Inc. A marker can be a passive IR reflector, an active IR emitter, an electromagnetic marker, and an optical marker used with an optical camera.

As is generally known within the art, implants and instruments may also be tracked by electromagnetic tracking systems. These systems locate and track devices and produce a real-time, three-dimensional video display of the surgical procedure. This is accomplished by using electromagnetic field transmitters that generate a local magnetic field around the patient's anatomy. In turn, the localization system includes magnetic sensors that identify the position of tracked instruments as they move relative to the patient's anatomy. By not requiring a line of sight with the transmitter, electromagnetic systems are also adapted for in vivo use, and are also integrable, for instance, with ultrasound and CT imaging processes for performing interventional procedures by incorporating miniaturized tracking sensors into surgical instruments. By processing transmitted signals generated by the tracking sensors, the system is able to determine the position of the surgical instruments in space, as well as superimpose their relative positions onto pre-operatively captured CT images of the patient.

Arrays 122 can be probe arrays, instrument arrays, reference arrays, calibrator arrays, and the like. Arrays 122 can have any number of markers, but typically have three or more markers to define real-time position (x, y, and z location) and orientation (rotation around x, y, and z axes). An array comprises a body and markers. The body comprises an area for spatial separation of the markers. In some embodiments, there are at least two arms and some embodiments can have three arms, four arms, or more. The arms are typically arranged asymmetrically to facilitate specific array and marker identification by the tracking system. In other embodiments, such as a calibrator array, the body provides sufficient area for spatial separation of markers without the need for arms. Arrays can be disposable or non-disposable. Disposable arrays are typically manufactured from plastic and include installed markers. Non-disposable arrays are manufactured from a material that can be sterilized, such as aluminum, stainless steel, and the like. The markers are removable, so they can be removed before sterilization.

Planning and collecting patient anatomical data 124 is a process by which a clinician inputs into the surgical navigation system actual or approximate anatomical data. Anatomical data can be obtained through techniques such as anatomic painting, bone morphing, CT data input, and other inputs, such as ultrasound and fluoroscope and other imaging systems.

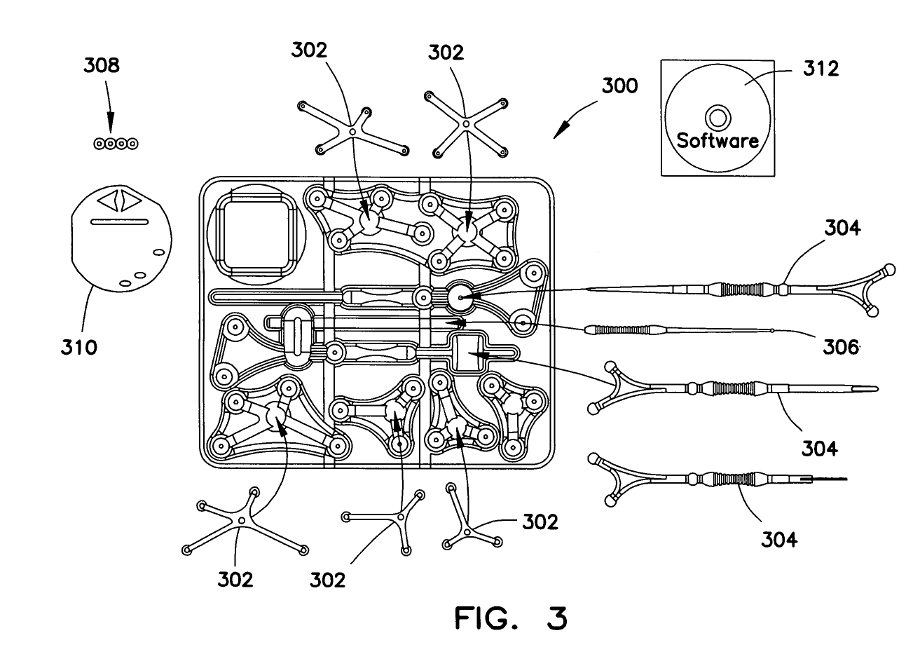

FIG. 3 shows orthopedic application kit 300, which is used in accordance with the present teachings. Application kit 300 is typically carried in a sterile bubble pack and is configured for a specific surgery. Exemplary kit 300 comprises arrays 302, surgical probes 304, stylus 306, markers 308, virtual keypad template 310, and application program 312. Orthopedic application kits are available for unicondylar knee, total knee, total hip, spine, and external fixation from EBI, L.P.

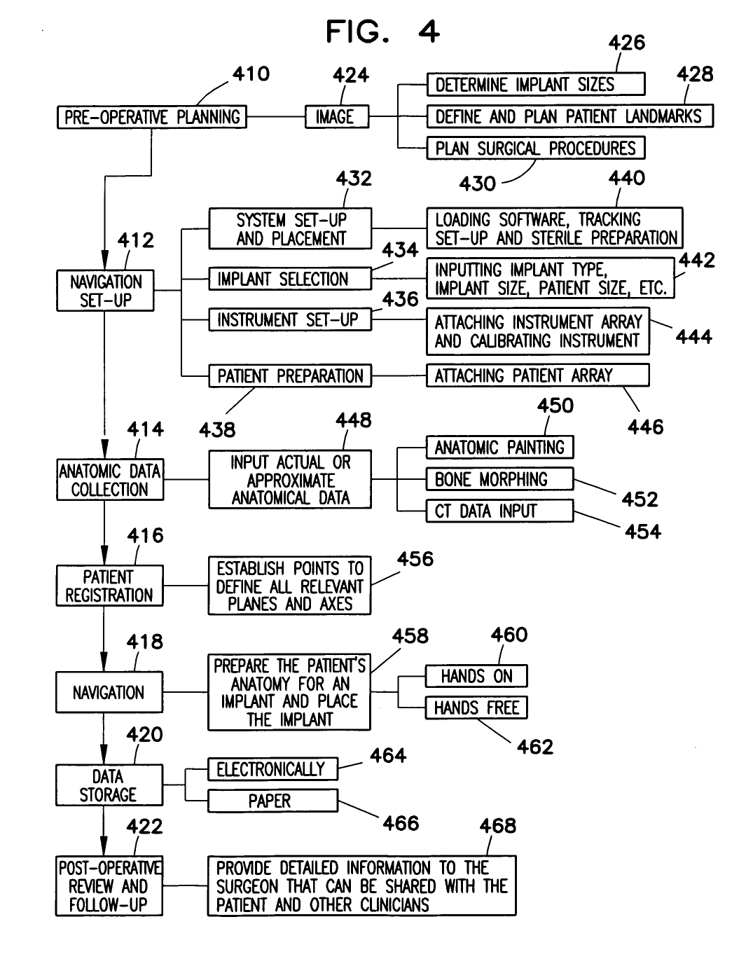

FIG. 4 shows an exemplary illustration of surgical navigation system 20. The process of surgical navigation according to this exemplary embodiment includes pre-operative planning 410, navigation set-up 412, anatomic data collection 414, patient registration 416, navigation 418, data storage 420, and post-operative review and follow-up 422.

Pre-operative planning 410 is performed by generating an image 424, such as a CT scan that is imported into the computer. With image 424 of the patient's anatomy, the surgeon can then determine implant sizes 426, such as screw lengths, define and plan patient landmarks 428, such as long leg mechanical axis, and plan surgical procedures 430, such as bone resections and the like. Pre-operative planning 410 can reduce the length of intra-operative planning thus reducing overall operating room time.

Navigation set-up 412 includes the tasks of system set-up and placement 432, implant selection 434, instrument set-up 436, and patient preparation 438. System set-up and placement 432 includes loading software, tracking set-up, and sterile preparation 440. Software can be loaded from a pre-installed application residing in memory, a single use software disk, or from a remote location using connectivity such as the internet. A single use software disk contains an application that will be used for a specific patient and procedure that can be configured to time-out and become inoperative after a period of time to reduce the risk that the single use software will be used for someone other than the intended patient. The single use software disk can store information that is specific to a patient and procedure that can be reviewed at a later time. Tracking set-up involves connecting all cords and placement of the computer, camera, and imaging device in the operating room. Sterile preparation involves placing sterile plastic on selected parts of the surgical navigation system and imaging equipment just before the equipment is moved into a sterile environment, so the equipment can be used in the sterile field without contaminating the sterile field.

Navigation set-up 412 is completed with implant selection 434, instrument set-up 436, and patient preparation 438. Implant selection 434 involves inputting into the system information such as implant type, implant size, patient size, and the like 442. Instrument set-up 436 involves attaching an instrument array to each instrument intended to be used and then calibrating each instrument 444. Instrument arrays should be placed on instruments, so the instrument array can be acquired by the tracking system during the procedure. Patient preparation 438 is similar to instrument set-up because an array is typically rigidly attached to the patient's anatomy 446. Reference arrays do not require calibration but should be positioned so the reference array can be acquired by the tracking system during the procedure.

As mentioned above, anatomic data collection 414 involves a clinician inputting into the surgical navigation system actual or approximate anatomical data 448. Anatomical data can be obtained through techniques such as anatomic painting 450, bone morphing 452, CT data input 454, and other inputs, such as ultrasound and fluoroscope and other imaging systems. The navigation system can construct a bone model with the input data. The model can be a three-dimensional model or two-dimensional pictures that are coordinated in a three-dimensional space. Anatomical painting 450 allows a surgeon to collect multiple points in different areas of the exposed anatomy. The navigation system can use the set of points to construct an approximate three-dimensional model of the bone. The navigation system can use a CT scan done pre-operatively to construct an actual model of the bone. Fluoroscopy uses two-dimensional images of the actual bone that are coordinated in a three-dimensional space. The coordination allows the navigation system to accurately display the location of an instrument that is being tracked in two separate views. Image coordination is accomplished through a registration phantom that is placed on the image intensifier of the C-arm during the acquisition of images. The registration phantom is a tracked device that contains imbedded radio-opaque spheres. The spheres have varying diameters and reside on two separate planes. When an image is taken, the fluoroscope transfers the image to the navigation system. Included in each image are the imbedded spheres. Based on previous calibration, the navigation system is able to coordinate related anterior and posterior views and coordinate related medial and lateral views. The navigation system can also compensate for scaling differences in the images.

Patient registration 416 establishes points that are used by the navigation system to define all relevant planes and axes 456. Patient registration 416 can be performed by using a probe array to acquire points, placing a software marker on a stored image, or automatically by software identifying anatomical structures on an image or cloud of points. Once registration is complete, the surgeon can identify the position of tracked instruments relative to tracked bones during the surgery. The navigation system enables a surgeon to interactively reposition tracked instruments to match planned positions and trajectories and assists the surgeon in navigating the patient's anatomy.

During the procedure, step-by-step instructions for performing the surgery in the application program are provided by a navigation process. Navigation 418 is the process a surgeon uses in conjunction with a tracked instrument or other tracked array to precisely prepare the patient's anatomy for an implant and to place the implant 458. Navigation 418 can be performed hands-on 460 or hands-free 462. However navigation 418 is performed, there is usually some form of feedback provided to the clinician such as audio feedback or visual feedback or a combination of feedback forms. Positive feedback can be provided in instances such as when a desired point is reached, and negative feedback can be provided in instances such as when a surgeon has moved outside a predetermined parameter. Hands-free 462 navigation involves manipulating the software through gesture control, tool recognition, virtual keypad and the like. Hands-free 462 is done to avoid leaving the sterile field, so it may not be necessary to assign a clinician to operate the computer outside the sterile field.

Data storage 420 can be performed electronically 464 or on paper 466, so information used and developed during the process of surgical navigation can be stored. The stored information can be used for a wide variety of purposes such as monitoring patient recovery and potentially for future patient revisions. The stored data can also be used by institutions performing clinical studies.

Post-operative review and follow-up 422 is typically the final stage in a surgical procedure. As it relates to navigation, the surgeon now has detailed information that he can share with the patient or other clinicians 468.

The present teachings enhance the above-described surgical navigation process by incorporating a bone milling procedure into surgical navigation system 20. Generally speaking, a surgical instrument used during a bone milling process is identified and tracked by the navigation system as it is moved relative to a patient's bone(s). The relative movement of the surgical instrument is detected by the tracking system and projected on a surgical plan image that is viewable by the surgeon. By tracking the relative movement of the surgical instrument on the plan image, the surgeon determines when the instrument is positioned at a location on the bone that is appropriate for performing the milling process (i.e., when the surgical instrument is located at the “surgical site”). The surgeon locates the surgical site by using a computer software program that is associated with the navigation system. The software program generates on-screen instructions that assist the surgeon in locating the surgical site as the surgical instrument is moved relative to the bone. More particularly, the software is programmed to locate the surgical site by referencing the patient's femoral mechanical axis, which connects the center of a patient's hip with the center of the patient's knee. The navigation system may also be programmed to locate a surgical site relative to a patient's tibial mechanical axis, which connects the center of the patient's knee with the center of the patient's ankle.

After the femoral mechanical axis is established, a line representing the axis is projected on a computer generated image of the bone so that the surgeon is able to position the surgical instrument at the surgical site. The surgeon is then prompted to make a hole in the bone with a drill or other similar surgical device. A guide pin is inserted into the hole, and a surgical tool is positioned over the guide pin to reshape the surface of the bone.

As these teachings allow the surgeon to place a guide pin with image-guided techniques, the use of referencing guides (e.g., “IM rods”) and/or other such invasive instruments is not required. More particularly, many conventional knee procedures involve the insertion of an IM rod into the bone marrow canal in the center of either the femur or tibia to assist in properly aligning the knee with the hip joint. However, because of the invasive application of such IM rods, patients are put at risk of developing fat embolism. More particularly, IM rods are capable of forcing body fat into a patient's blood stream. If this happens, the fat deposit may become lodged in the patient's heart or brain and cause the patient to suffer from heart failure, dementia or stroke. As these teachings do not require such invasive measures, the risk of fat embolism is reduced. Moreover, as the present methods do not require the surgeon to administer a large incision at the surgical site, the patient's recovery time is also improved.

Turning now to a more detailed discussion of the present teachings, and referring again to FIG. 1, drill guide 31 includes tracking array 35, which is detectable by the tracking system. When drill guide 31 is moved relative to patient 22, the movement is captured by the tracking system and projected on surgical plan image 29 of computer monitor 27. Surgical plan image 29 depicts a graphical representation of the patient's bones and shows in real-time the position of drill guide 31 as it moves relative to the patient's bones. Surgeon 21 views plan image 29 and determines when drill guide 31 is positioned at the targeted surgical site. Once drill guide 31 is positioned at the surgical site, surgeon 21 drills a hole into the bone and places a guide pin into the hole. Surgeon 21 then positions a surgical tool, such as a reamer, directly over the inserted guide pin and mills or removes a portion of the bone.

While the above illustration uses a tracked drill guide to place the guide pin at the surgical site, a drill guide is not required in other alternative embodiments. For instance, the guide pin and surgical reamer can be coupled together to form a unitary surgical device, such as a reamer with a drill guide tip. According to this illustration, the tracking system detects the surgical device as it is moved relative to the patient's bones and graphically displays the movement on the surgical plan image. Once the surgeon locates the device at the surgical site, the drill guide is advanced into the bone and upon contact with the bone, begins to mill a portion of the bone.

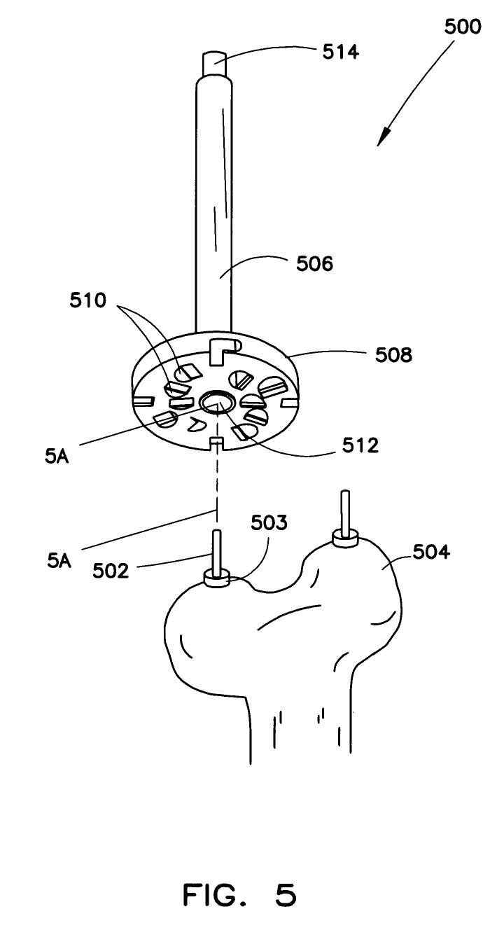

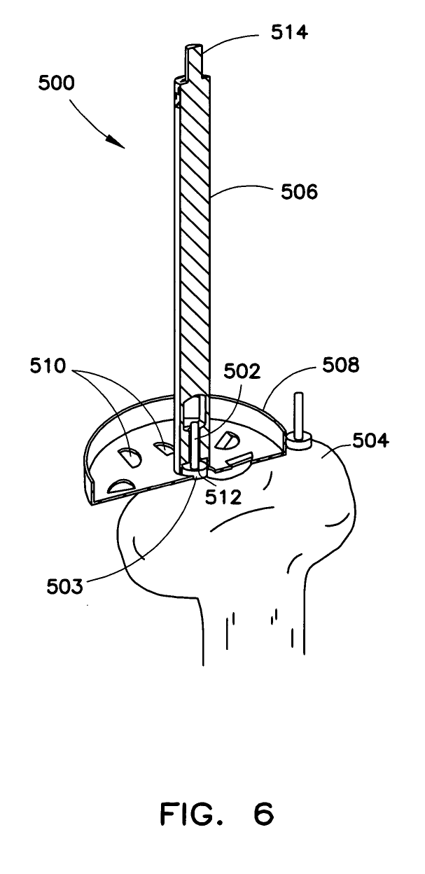

An exemplary surgical reamer 500 is shown in FIGS. 5 and 6. Surgical reamer 500 includes elongated shaft 506, cutting plate 508, and coupling member 514 for releasably attaching the reamer to a variety of surgical instruments, such as surgical drill 32 shown in FIG. 1. Cutting plate 508 includes a plurality of cutting blades 510, as well as bore 512. To mill bone 504, cutting plate 508 is engaged with the surface of the bone by inserting reamer 500 over guide pin 502 along line 5A-5A. In other words, bore 512 receives guide pin 502 such that cutting plate 508 is pressed against the surface of bone 504 (as best shown in FIG. 6). In certain illustrations, bore 512 can function as a stop surface that controls the depth to which cutting plate 508 penetrates the bone during the milling process. In alternative illustrations, a shaft of the guide pin controls the depth to which the bone is penetrated by cutting plate 508. To accomplish this, the shaft includes stop collar 503 that affects the depth to which the blades of the cutting plate are permitted to penetrate the bone during the resection process.

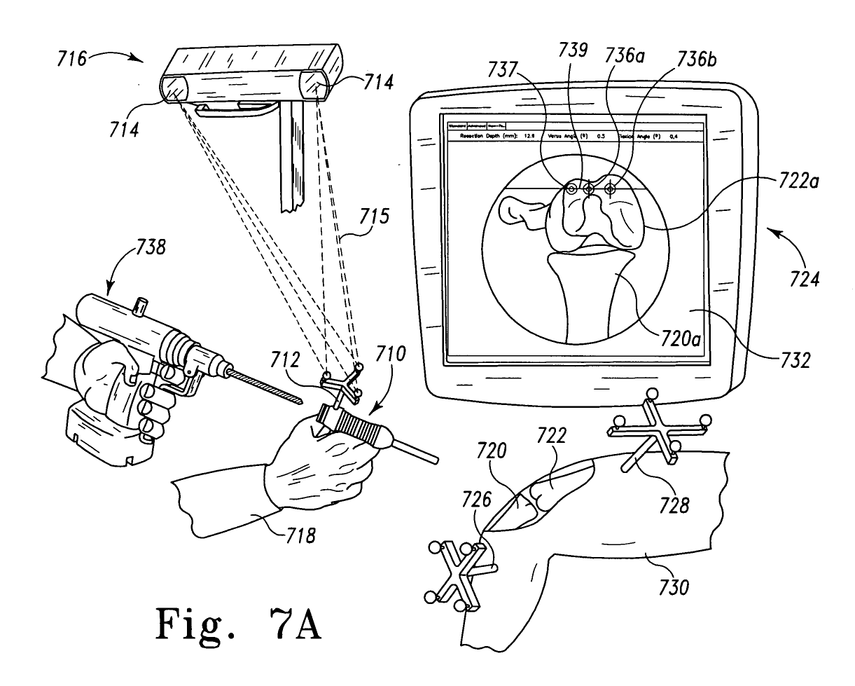

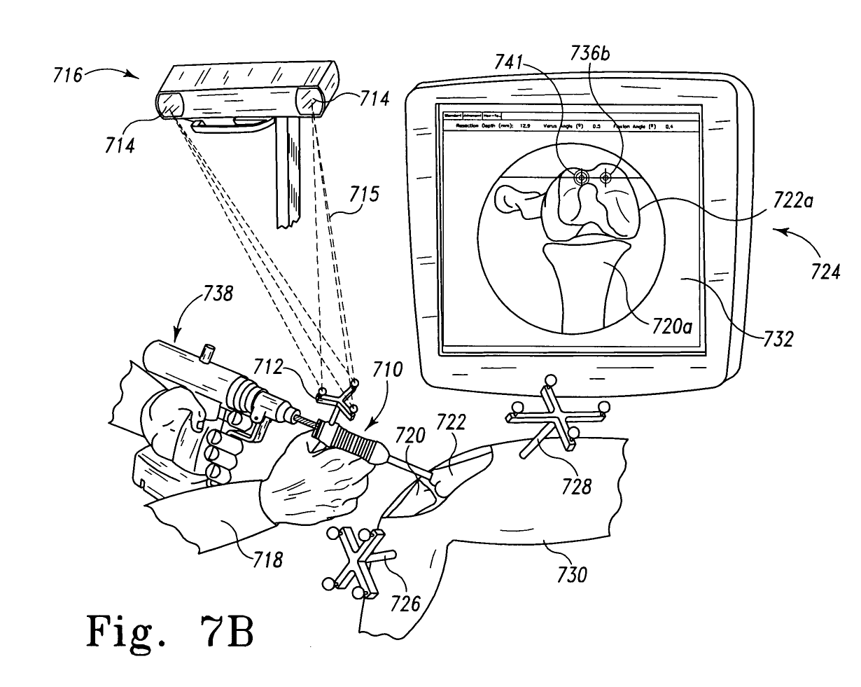

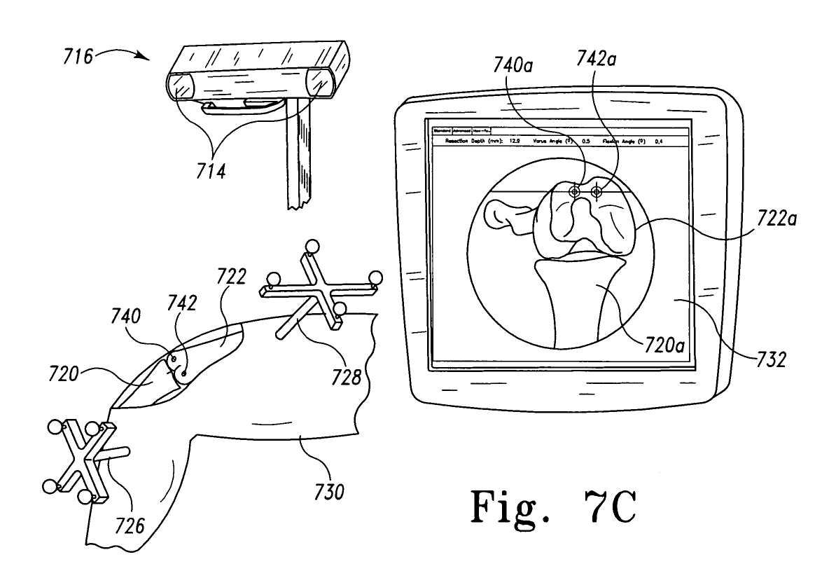

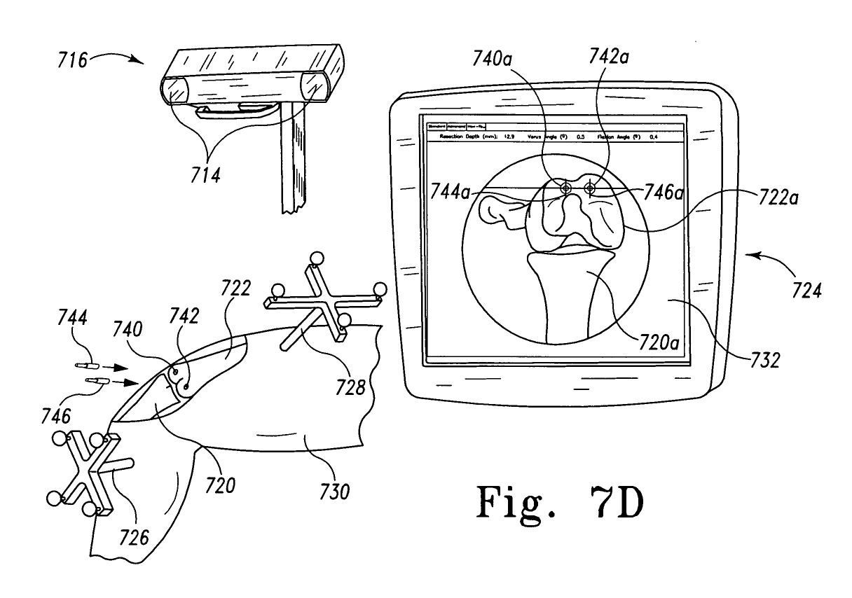

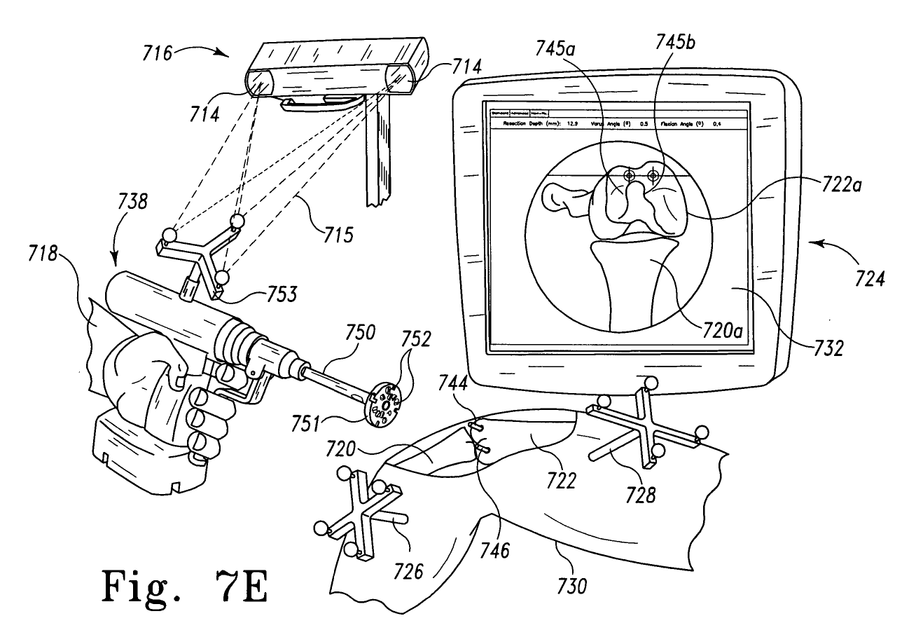

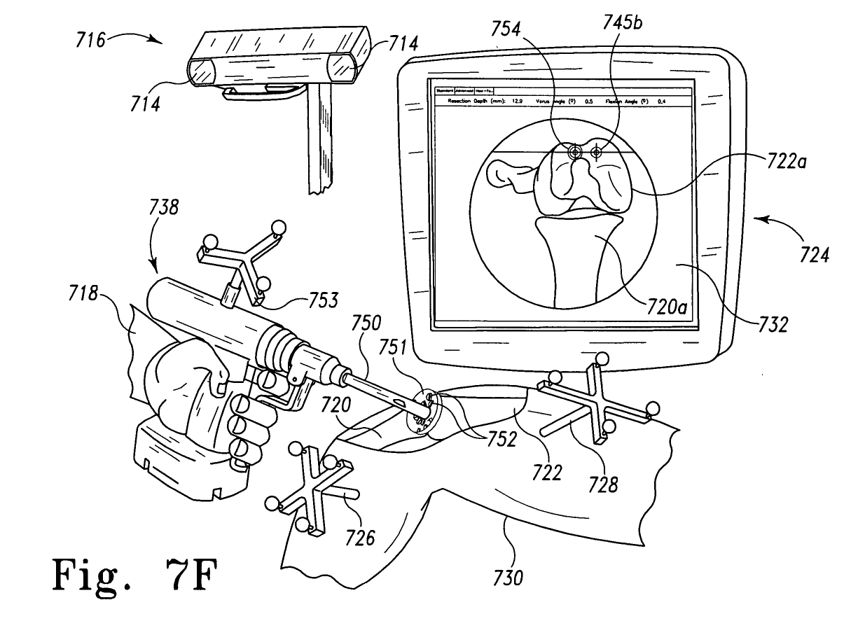

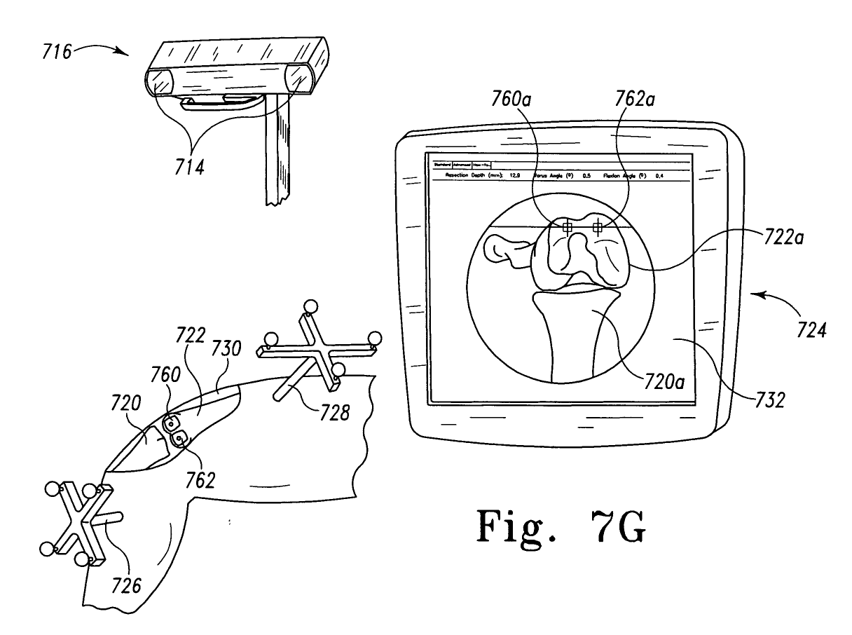

An exemplary illustration of a bone undergoing a milling process in accordance with the present teaching is depicted in FIGS. 7A-7G. Drill guide 710 includes marker array 712, which is identified and tracked by cameras 714 of optical locator 716. As surgeon 718 moves drill guide 710 relative to bones 720 and 722, the tracking system locates and tracks marker array 712 in real-time (see the optical path/measurement field of the tracking system represented by dashed lines 715). To accomplish this, cameras 714 of optical locator 716 detect the position of marker array 712 in space by using triangulation methods. The relative location of marker array 712 is then shown on surgical plan image 732 on computer display 724.

The tracking system detects the location of drill guide 710 relative to bones 720, 722 by referencing the position of marker array 712 as it moves with respect to reference arrays 726 and 728, which are fixably attached to the tibia and femur of patient 730. As shown in FIG. 7A, the position of drill guide 710 is displayed on surgical plan image 732 as drill location icon 737. According to this illustration, drill location icon 737 is shown positioned over the distal condyle of surgical bone 722, such that drilling will occur from distal to proximal on the distal condyle of bone 722. By viewing drill location icon 737 on surgical plan image 732, surgeon 718 determines which direction to move drill guide 710 so that it aligns with either of surgical target sites 736a or 736b on surgical bone images 720a, 722a (which respectively correspond to bones 720, 722). For instance, in this illustrated embodiment, surgeon 718 must move drill guide 710 immediately to the right along line 739 to align drill location icon 737 with surgical target site 736a. To locate surgical target site 736a, the distal most point on the distal medial femoral condyle may be referenced by the surgeon and/or the computer tracking system. In certain exemplary embodiments, surgical target site 736a is identified by modeling the medial distal femoral condyle through a “painting” or imaging technique which allows the computer system to determine the distal most point on bone 722. In further exemplary embodiments, the surgical target site is identified by referencing the patient's femoral mechanical axis, which connects the center of the patient's hip with the center of the patient's knee. In this embodiment, the navigation system's software identifies the mechanical axis and projects its image on a computer generated image of the femur. No matter how surgical site 736a is determined, however, if it is later found to be inappropriate for conducting the surgery (i.e., too medial or central), surgeon 718 is always able to override the site and rely on the computer for orientation only (parallel to the mechanical axis).

As shown in FIG. 7B, once drill guide 710 is located at surgical target site 736a, drilling target 741 appears on surgical plan image 732 thereby prompting surgeon 718 to drill into bone 722 with surgical drill 738. As surgeon 718 aligns drill guide 710 with surgical target site 736a by using surgical navigation technology, invasive instruments and/or IM referencing guides are avoided. Accordingly, patient 730 has a reduced risk of developing an embolism, as explained above.

As shown in FIG. 7C, after surgeon 718 drills into bone 722 with surgical drill 738, surgical plan image 732 shows hole image 740a on bone image 722a, which directly corresponds to hole 740 on bone 722. Surgeon 718 then inserts surgical guide pin 744 into hole 740 and confirms its insertion with software associated with the tracking system. The surgical plan image 732 then shows the inserted pin as image 744a in hole image 740a (see FIG. 7D). Once surgical guide pin 744 is inserted into hole 740, surgeon 718 prepares to resurface or ream bone 722 with surgical reamer 750, which is affixed to surgical drill 738 (see FIG. 7E). To accomplish this, surgeon 718 positions surgical reamer 750 over guide pin 744 at the resurface site shown on surgical plan image 732 as site 745a and engages bone 722 with cutting plate 751 (see FIG. 7F). Once reamer 750 is positioned over guide pin 744, surgical plan image 732 shows the position of the reamer relative to the bone as reamer locator icon 754.

By activating surgical drill 738, cutting plate 751 of reamer 750 rotates and causes cutting blades 752 to penetrate the bone and create planar surface 760, which corresponds to reamed surface image 760a on surgical plan image 732 (see FIG. 7G). To assist in creating planar surface 760, drill array 753 is coupled to surgical drill 738, and is identified and tracked by the tracking system. The tracking system recognizes the position of reamer 750 as it moves relative to bone 722 and can thereby determine how much bone is removed as the surface of the bone is reamed. Moreover, software associated with the navigation system can be programmed to generate real-time instructions to the surgeon during the resurfacing process so that the surgeon has an accurate reading of how much bone has been removed and/or still needs to be removed before affixing the implant to the cut bone.

Depending on the surgical procedure to be performed, the present teachings allow for more than one planar surface to be created on bone 722. For instance, total knee procedures require that both condyles be resurfaced. To accomplish this, surgeon 718 positions drill guide 710 at surgical target site 736b and drills hole 742 into bone 722, which corresponds to hole image 742a on surgical plan image 732 (see FIGS. 7A-7C). After drilling hole 742, surgeon 718 places guide pin 746 into the hole, and surgical plan image 732 shows the inserted pin as image 746a (see FIGS. 7D-7E). Surgical reamer 750 is then positioned over guide pin 746 at the resurface site shown as 745b on plan image 732 and engages the surface of bone 722 with cutting plate 751 to create planar surface 762, which corresponds to reamed surface image 762a on surgical plan image 732 (see FIGS. 7F-FG).

In the above example of the inventive method, surgeon 718 has created two co-planar surfaces on the femoral condyles of bone 722. According to this embodiment, the tracking system, via tracking the drill guides, orients both planes perpendicular to the mechanical axis. The system, by tracking the reamer, can also assist the surgeon with determining the reaming depth thereby ensuring that the two planes are co-planar. However, one of ordinary skill would readily recognize that the present teachings may be used with multiple bone resurfacing procedures, including both uni-knee and total knee operations, as well as patellofemoral resurfacing procedures and/or any other procedures requiring the creation of a milled geometry on the bone.

The methods of resurfacing a bone at a surgical site during a surgical navigation procedure according to the present teachings can also be embodied on a computer readable storage medium. According to these embodiments, the computer readable storage medium stores instructions that, when executed by a computer, cause the surgical navigation system to perform a bone resurfacing process at a surgical site. The computer readable storage medium can be any medium suitable for storing instruction that can be executed by a computer such as a compact disc (CD), digital video disc (DVD), flash solid-state memory, hard drive disc, floppy disc, and the like.

While exemplary embodiments incorporating the principles of the present teachings have been disclosed hereinabove, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain and which fall within the limits of the appended claims.

What is claimed is:

1. A method of resurfacing a bone at a surgical site during a surgical navigation procedure, comprising:
providing a tracking system and a surgical instrument having a tracking array, the tracking array being identified and tracked by the tracking system;
moving the surgical instrument relative to the bone while the tracking system tracks the position of the surgical instrument, the relative movement being projected on an image of the bone;
viewing the projected image as the surgical instrument is moved relative to the bone to determine when the surgical instrument is positioned at the surgical site;
using the surgical instrument to make a hole in the bone at the surgical site; and
inserting a guide pin into the hole.

2. The method of claim 1, further comprising:
positioning a surgical tool over the guide pin; and
milling a first portion of the bone with the surgical tool to create a first substantially planar surface on the bone.

3. The method of claim 2, further comprising:
using the surgical instrument to make a second hole in the bone at the surgical site;
inserting a second guide pin into the second hole;
positioning the surgical tool over the second guide pin; and
milling a second portion of the bone with the surgical tool to create a second substantially planar surface on the bone, the second surface being substantially coplanar with the first surface.

4. The method of claim 1, wherein the moving of the surgical instrument relative to the bone comprises moving a drill guide.

5. The method of claim 1, wherein the moving of the surgical instrument relative to the bone comprises moving a drill.

6. The method of claim 1, wherein the viewing of the projected image comprises viewing a real-time graphical image of the bone.

7. The method of claim 2, wherein the positioning of the surgical tool over the guide pin comprises positioning a reamer over the guide pin.

8. The method of claim 1, further comprising:
locating the surgical site by referencing a mechanical axis, the mechanical axis being identified by the tracking system.

9. The method of claim 8, wherein the mechanical axis connects the center of a patient's hip with the center of the patient's knee.

10. The method of claim 1, wherein the procedure is performed without a referencing guide.

11. The method of claim 10, wherein the performance of the procedure without a referencing guide comprises performing the procedure without an intramedullary rod.

12. The method of claim 1, wherein the resurfacing of the bone comprises resurfacing a femur.

13. A method of performing a knee procedure using surgical navigation, comprising:
providing a tracking system and a surgical instrument having a tracking array, the tracking array being identified and tracked by the tracking system;
using the tracking system to guide the surgical instrument to a surgical site on a patient's femur;
using a surgical tool to create a first substantially planar surface on a condyle of the patient's femur at the surgical site; and
installing a surgical implant on the femur;
wherein the procedure is performed without a referencing guide.

14. The method of claim 13, further comprising:
determining the location of the surgical site by referencing a mechanical axis identified by the tracking system, the mechanical axis connecting the center of a patient's hip with the center of the patient's knee.

15. The method of claim 14, wherein a line representing the mechanical axis is projected on a computer generated image of the femur.

16. The method of claim 14, wherein the mechanical axis comprises a femoral mechanical axis.

17. The method of claim 13, further comprising:
making a hole in the condyle at the surgical site; and
inserting a guide pin into the hole.

18. The method of claim 17, wherein using the surgical tool to create the first substantially planar surface on the condyle comprises positioning the surgical tool over the guide pin and milling a first portion of the condyle with the surgical tool.

19. The method of claim 18, further comprising:
making a second hole in the condyle at the surgical site;
inserting a second guide pin into the second hole
positioning the surgical tool over the second guide pin; and
milling a second portion of the condyle with the surgical tool to create a second substantially planar surface on the condyle, the second surface being substantially coplanar with the first surface.

20. The method of claim 13, wherein the guiding of the surgical instrument to the surgical site comprises guiding a drill guide.

21. The method of claim 13, wherein the guiding of the surgical instrument to the surgical site comprises guiding a drill.

22. The method of claim 13, wherein the use of a surgical tool to create a first substantially planar surface on the condyle comprises using a reamer.

23. The method of claim 13, wherein the performance of the procedure without a referencing guide comprises performing the procedure without an intramedullary rod.

24. The method of claim 13, wherein the knee procedure comprises a knee arthroplasty procedure.

25. A computer readable storage medium for use with a surgical navigation system, the storage medium storing instructions that, when executed during a bone resurfacing procedure, cause the surgical navigation system to implement the following steps:
tracking a surgical instrument having a tracking array with a tracking system as the surgical instrument moves relative to the bone;
projecting the relative movement of the surgical instrument on an image of the bone;
displaying a mechanical axis on the image, the mechanical axis being positioned relative to the surgical site;
identifying a location on the image having a corresponding location on the bone for inserting a guide pin once the surgical instrument is positioned at the surgical site; and
generating instructions for creating a first substantially planar surface on a first portion of the bone at the location of the first guide pin.

26. The computer readable storage medium of claim 25, wherein the stored instructions, when executed, further comprise causing the surgical navigation system to identify a second location on the image having a corresponding location on the bone for inserting a second guide pin to create a second substantially planar surface on a second portion of the bone at the location of the second guide pin.

27. The computer readable storage medium of claim 26, wherein the stored instructions, when executed, further cause the surgical navigation system to generate instructions for creating the second substantially planar surface on the second portion of the bone at the location of the second guide pin.

28. The computer readable storage medium of claim 25, wherein the stored instructions, when executed, further comprise causing the surgical navigation system to determine the location of the surgical site by referencing the mechanical axis identified by the tracking system, the mechanical axis connecting the center of a patient's hip with the center of a patient's knee.

29. The computer readable storage medium of claim 25, wherein the mechanical axis comprises a femoral mechanical axis.

30. The computer readable storage medium of claim 25, wherein the bone comprises a femur.
